(19) United States
(12) Patent Application Publication
(10) Pub. No.: US 2006/0147536 A1
(43) Pub. Date: Jul. 6, 2006

(54) Method for removing fluids containing dissolved or suspended carbohydrates, lipids, metals, salts, and/or toxins from biological systems

(76) Inventor: Robert A. Fiore, East Longmeadow, MA (US)

Correspondence Address: HOLLAND & BONZAGNI, P.C., 171 DWIGHT ROAD, SUITE 302, LONGMEADOW, MA, 01106-1700, US

(21) Appl. No.: 11/026,072

(22) Filed: Dec. 30, 2004

(51) Int. Cl. A61K 9/14

(52) U.S. Cl. 424/489

(57) ABSTRACT

A means for reducing the levels of carbohydrates, lipids, metals, salts and/or toxins in biological systems thereby preventing or reducing weight gain, while reducing, for example, the incidence of atherosclerosis, hypertension, and toxic reactions, is provided. More specifically, the subject invention provides non-toxic, substantially non-degradable, optionally collapsible and/or expandable particles to react or complex with, decompose, absorb and/or adsorb dissolved or suspended carbohydrates, lipids, metals, salts and/or toxins that are present in fluids found in a target biological system.

### TECHNICAL FIELD OF THE INVENTION

The present invention basically relates to a means for reducing the levels of carbohydrates, lipids, metals, salts and/or toxins in biological systems thereby preventing or reducing weight gain, while reducing, for example, the incidence of atherosclerosis, hypertension, and toxic reactions. More specifically, the subject invention contemplates the use of non-toxic, substantially non-degradable, optionally collapsible and/or expandable particles to react or complex with, decompose, absorb and/or adsorb dissolved or suspended carbohydrates, lipids, metals, salts and/or toxins that are present in fluids found in a target biological system.

### BACKGROUND OF THE INVENTION

A growing incidence of obesity, atherosclerosis and hypertension has been observed within the developed world and in particular the United States. For many years, invasive and non-invasive techniques have been used to treat these conditions.

Certain consumed substances including fats, salts and sugars, have been identified as contributing to the above named conditions, and as such a viable non-invasive technique has been developed that serves to lower the levels of these substances in biological systems such as the digestive system.

Prior art attempts to remove problematic materials from the digestive tract typically target a specific material or type of material and rely solely upon adsorptive means to bind and thereafter remove the material(s) from the digestive tract.

For example, U.S. Pat. No. 6,638,498 B2 relates to novel molecularly imprinted polymers or MIPs that bind to, and thereby remove, toxins (e.g., bile acids, bile salts) in the gastrointestinal tract. The MIPs may be administered to humans orally by way of, for example, powders or granules, capsules or tablets.

By way of further example, U.S. Pat. No. 5,514,281 relates to an adsorbent material modified with polynuclear metal oxyhydroxides that is used for the selective removal of inorganic phosphate from e.g. the liquid contents of the intestine. The adsorbent material may be administered in the form of an oral preparation. More specifically, the adsorbent material may be pressed into a powder and either coated with a layer resistant to gastric acid or dispensed into an acid-resistant capsule.

In view of the above, it is an object of the present invention to provide a means for removing problematic materials from biological systems that relies upon several different removal techniques or mechanisms, and that preferably targets more than one such material for removal or elimination. The subject invention offers significant advantages when compared to prior art procedures.

### SUMMARY

The present invention therefore provides non-toxic, substantially non-degradable, optionally collapsible and/or expandable particles for use in reducing levels of carbohydrates, lipids, metals, salts and/or toxins in biological systems such as the digestive system, that each comprise an optionally bifurcated core that is at least partially surrounded by an impermeable, semi-permeable, or permeable membrane or membrane-like material.

The present invention further provides a method of reducing levels of carbohydrates, lipids, metals, salts and/or toxins in biological systems such as the digestive system, which basically comprises: delivering a plurality of the above-described particles to the target system.

In one embodiment contemplated by way of the present invention, the inventive particles each comprise:

(a) a solid or semi-solid core comprising materials capable of reacting or complexing with, decomposing, absorbing and/or adsorbing dissolved or suspended carbohydrates, lipids, metals, salts and/or toxins; that is at least partially surrounded by

(b) an optionally expandable, impermeable, semi-permeable, or permeable membrane or membrane-like material.

In another contemplated embodiment, the inventive particles each comprise:

(a) a hollow core; that is at least partially formed by

(b) an optionally collapsible and/or expandable, impermeable, semi-permeable, or permeable membrane or membrane-like material.

In yet another contemplated embodiment, the inventive particles each comprise a bifurcated core having a first portion and a second portion, wherein the bifurcated core is at least partially surrounded or formed by an optionally collapsible and/or expandable, impermeable, semi-permeable, or permeable membrane or membrane-like material, and wherein the first portion of the bifurcated core contains buoyancy-enhancing or effervescent agents.

In yet a further embodiment, the inventive particles are either compressed into tablet form or contained within a dissolvable or erodible polymeric carrier (e.g., protective capsule).

Other features and advantages of the invention will be apparent to one of ordinary skill from the following detailed description and accompanying drawings.

Unless otherwise defined, all technical and scientific terms used herein have the same meaning as commonly understood by one of ordinary skill in the art to which this invention belongs. All publications, patent applications, patents and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

Particular features of the disclosed invention are illustrated by reference to the accompanying drawings in which:

FIG. 1 is a cross-sectional view of the non-toxic, substantially non-degradable, optionally collapsible and/or expandable particle of the present invention;

FIGS. 2 to 4 are cross-sectional views of preferred embodiments of the inventive particle, each having a solid or semi-solid core;

FIGS. 5 to 7 are cross-sectional views of other preferred embodiments of the inventive particle, each having a hollow core; and

FIG. 8 is a cross-sectional view of yet another preferred embodiment of the inventive particle that has a bifurcated core.

### BEST MODE FOR CARRYING OUT THE INVENTION

By way of the subject invention, fluids containing suspended lipids, dissolved salts and sugars, metals (e.g., various heavy metals) and/or toxins may be removed from biological systems such as the digestive system (i.e., the gastrointestinal tract which includes the stomach and lower gastrointestinal tract), and the circulatory, endocrine, excretory, lymphatic, integumentary, respiratory and musculatory systems.

The subject invention may form part of weight control and reduction programs due to the ability of the inventive particles to react or complex with, decompose, partially absorb and/or adsorb, for example, dissolved, high caloric lipids and sugars, and in a preferred embodiment, further due to its ability to convey a so-called “full-stomach feeling” to individuals participating in such a program due to simple volumetric expansion of the inventive particles upon ingestion.

The subject invention may also play a part in the management and control of hypertension due to its ability to absorb and/or adsorb dissolved salts.

Additional areas in which the subject invention may be used include, but are not limited to, the treatment of individuals affected by toxins by the select removal of these toxins in both emergency and non-emergency situations, the treatment of individuals with a history of alcohol and/or drug abuse by the select removal of the abused substance or substances, and the treatment of individuals with varying degrees of atherosclerosis by the select removal of various heavy metals such as copper and iron.

As noted above, the inventive particles comprise an optionally bifurcated core that is at least partially surrounded or formed by an impermeable, semi-permeable, or permeable membrane or membrane-like material. These particles are preferably made from materials regarded as safe for human consumption and as such are non-toxic (i.e., biocompatible). The inventive particles are preferably also substantially non-degradable (i.e., acid resistant).

The terms “non-toxic” and “biocompatible”, as used herein, mean that when ingested or injected in recommended dosages or amounts, the inventive particles do not produce a toxic, injurious, or immunological response in living tissue, while the terms “non-degradable” and “acid resistant” means that the inventive particles will not substantially hydrolyze, or oxidize, or otherwise decompose or degrade for a period of time ranging from about 4 to about 72 hours when exposed to biological agents (e.g., bacteria) and acids found in target biological systems.

Referring now to the drawings in detail, the non-toxic and substantially non-degradable particle of the present invention is shown and generally designated by reference numeral 10.

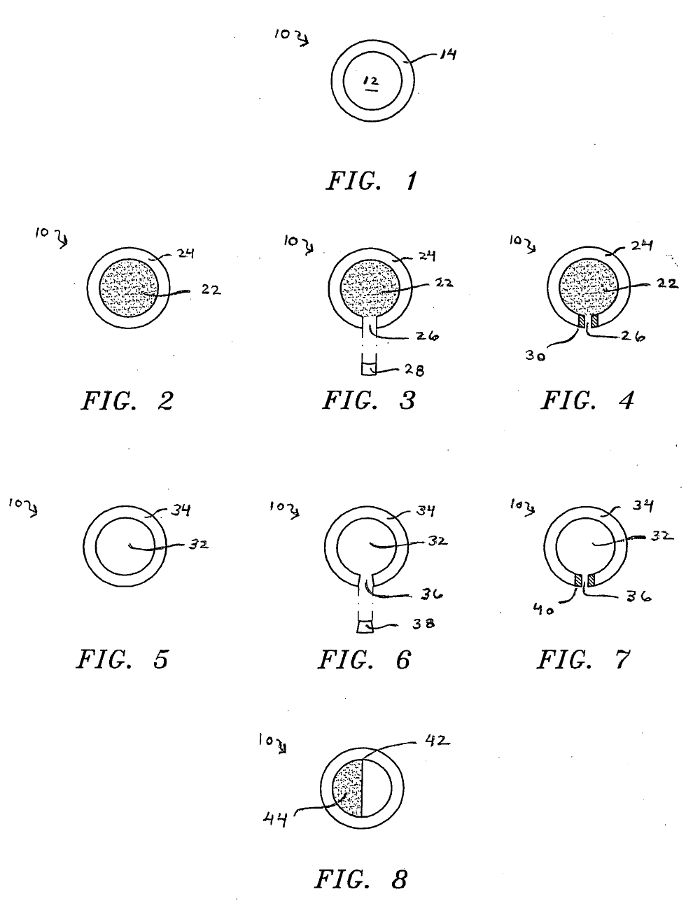

As best shown in FIG. 1, the inventive particle basically comprises: a core 12; that is at least partially surrounded by an optionally collapsible and/or expandable, thin sheet or wall of natural or synthetic material (i.e., membrane) 14.

As will be described in more detail below, the core 12 may be a solid, semi-solid or hollow core, while the membrane 14 may be impermeable, permeable, or semi-permeable, and prepared from, for example, non-degradable polymers, ceramics, glasses, metals, or similar materials.

Suitable non-degradable polymers for use in preparing membrane 14 include cellulose esters, cellulose ethers, cellulose esters-ethers, cellulose acetate, acyl substituted cellulose acetate and derivatives thereof, chole-styramine, ethylene-vinyl acetate polymers, non-degradable polyurethanes, polyacrylates, polyamides, polyethylene, poly(ethylene glycol), polyethylene oxide, poly(N-vinylpyrrolidone), chlorosulphonated polyolefins, polystyrenes, polytetrafluoroethylene (PTFE), polyvinyl alcohol, polyvinyl chloride, polyvinyl fluoride, poly(vinyl imidazole) and silicone, while suitable ceramic, glass, and metal materials include metal oxides such as alumina, mullite, cordierite, zircon and zirconia, synthetically produced and naturally occurring glasses, metals in the form of dispersed metal particles, metal glasses such as metal glass alloy compositions, and combinations thereof.

The inventive particles 10 may be manufactured using conventional methods and techniques, such as those described in U.S. Pat. Nos. 5,073,612, 6,669,961, 6,290,983, and 6,103,271.

While the shape and size of the inventive particles are not limited, the particles are preferably substantially spherical particles having a diameter of less than about 5 millimeters (mm). In a preferred embodiment, the inventive particles are micro- or nano-sized particles, with the micro-sized particles having a diameter ranging from about 1 to about 2,000 micrometers or microns (μm) (preferably, from about 10 to about 1,000 μm), and with the nano-sized particles having a diameter ranging from about 1 to about 1,000 nanometers (nm) (preferably, from about 50 to about 500 nm).

In several contemplated embodiments, which are best shown in FIGS. 2 to 4, particle 10 basically comprises: a solid or semi-solid core 22, which is at least partially surrounded by either an impermeable, permeable, or semi-permeable membrane 24.

A wide variety of materials may be used to form solid or semi-solid core 22, which serve to react or complex with, decompose, absorb and/or adsorb dissolved or suspended carbohydrates, lipids, metals, salts, and/or toxins present in fluids found in a target environment. Contemplated active core materials include microorganisms including active and pathological bacteria (e.g., bacterium Rhodoferax ferrireducens), catalysts, and enzymes. Adsorbent and/or absorbent solid and semi-solid core materials contemplated by way of this invention include absorbent and superabsorbent polymers (e.g., sodium polyacrylate) and resins (e.g., resinous chelating agents including styrene divinylbenzene copolymer, derivatives of iminodiacetic acid such as EDTA, hydrocolloids, matrix bound glycine hydroxamic acid), natural sponges, synthetic sponges (e.g., porous plastics, rubber, cellulose), carbon, activated carbon, ceramics, clays, oatmeal and other plant-derived materials, silicas, natural and synthetic silicates, silica gels, silicon, and combinations thereof. These materials may be used alone or combined with one or more sequestering agents, such as ethylene diamine tetra-acetic acid, acidic acid, boric acid, citric acid, gluconic acid, lactic acid, phosphoric acid, tartaric acid, and salts thereof, metaphosphates, dihydroxyethylglycine, lecithin, beta cyclodextrin, and combinations thereof.

As will be readily appreciated by those skilled in the art, the core materials may take a number of different forms including microbeads or microspheres, fibers, hollow fibers or tubelets, and may swell or expand upon absorption and/or adsorption of dissolved or suspended materials found in the target fluids.

Materials suitable for use in forming membrane 24 are described above.

Referring now to FIG. 2, particle 10 is shown as comprising a solid or semi-solid core 22 that is completely surrounded by semi-permeable or permeable membrane 24. In this embodiment, fluid is drawn into core 22 via simple osmotic action. In FIGS. 3 and 4, solid or semi-solid core 22 is shown as being partially surrounded by either an impermeable, semi-permeable, or permeable membrane 24 that is provided with at least one aperture or hole 26 that communicates directly with core 22. In these embodiments, fluid is drawn into core 22 by way of a combination of simple osmotic and capillary action.

As shown in FIG. 3, aperture 26 may be provided with an erodable stopper or plug 28 that serves to prevent or retard capillary movement of fluid into or out of solid or semi-solid core 22 until the particle 10 is properly positioned within the target physiological environment. Suitable materials for use in preparing erodable stopper or plug 28 include, but are not limited to, materials that are insoluble or substantially insoluble in water such as waxes.

As shown in FIG. 4, aperture 26 may contain a swellable material 30 that swells or increases in size when exposed to fluids found in the target environment, thereby containing or sequestering acquired fluids within core 22. Swellable materials 30 contemplated by way of the present invention include, but are not limited to, alginic acid, calcium alginate, calcium pectinate, calcium phosphate, carboxymethylcellulose, carrageenan, cellulose, crosprovidone, cross-linked guar gum, cross-linked hydrophilic polymer, cross-linked polysaccharide, cross-linked vegetable guar gum, cross-linked vegetable gum, gelatins, gum and agar, microcrystalline cellulose, microcrystalline starch, natural clays, polyacrylates, polymer crystals, polyvinyl pyrrolidone, sodium alginate, sodium polyacrylate, starch, and combinations thereof.

In other contemplated embodiments, which are best shown in FIGS. 5 to 7, particle 10 basically comprises: a hollow core 32 that is at least partially formed by either an impermeable, semi-permeable, or permeable membrane 34.

In FIG. 5, hollow core 32 is formed by semi-permeable or permeable membrane 34, with fluid being drawn into hollow core 32 via simple osmotic action. In FIGS. 6 and 7, hollow core 32 is formed by either an impermeable, semi-permeable, or permeable membrane 34 that is provided with at least one aperture or hole 36 that communicates directly with hollow core 32. In these embodiments, fluids are drawn into hollow core 32 by way of a combination of simple osmotic and capillary action.

As shown in FIG. 6, aperture 36 may be provided with an erodable stopper or plug 38 that serves to prevent or retard capillary movement of fluid into and out of hollow core 32 until the particle 10 is properly positioned within the target physiological environment.

As shown in FIG. 7, aperture 36 may contain a swellable material 40 that swells or increases in size when exposed to fluids found in the target environment, thereby containing or sequestering acquired fluids within hollow core 32.

The rate at which fluid is drawn into hollow core 32 may be increased by generating or forming a partial vacuum within particle 10. A partial vacuum may be formed within hollow particle 10 using known methods or techniques which include:

(1) manufacturing the inventive particles 10 at sub-atmospheric pressures to form collapsed or flattened particles, and placing these collapsed or flattened particles in a pressurized and/or volume restricted polymeric containment vessel or carrier. In a preferred embodiment, the particles 10 are manufactured using shape-memory polymers; and

(2) collapsing or flattening the inventive particles 10 post manufacture using, for example, mechanical, chemical, electrical (e.g., piezoelectric, pyroelectric) and/or thermal means, and then placing these collapsed or flattened particles in a pressurized and/or volume restricted polymeric containment vessel. As above, the particles 10 are preferably manufactured using shape-memory polymers.

Upon release of the pressure exerted upon particle 10 (i.e., upon release from the pressurized or volume restricted polymeric containment vessel or capsule), the particle would return to its original shape thereby forming a partial vacuum within particle 10 that serves to draw fluid toward and within core 12.

As alluded to above, and as best shown in FIG. 8, particle 10 of the present invention may employ a bifurcated core 42, a portion of which contains buoyancy-enhancing (i.e., effervescent) agents 44. The inventive particle 10 may also be joined to one or more secondary buoyancy-enhancing particles containing these agents.

Contemplated buoyancy-enhancing or effervescent agents 44 include compounds which evolve gas, with preferred agents evolving gas by means of a chemical reaction that takes place upon exposure of the agent to fluids (e.g., water) found in the target environment. This reaction is most often the result of the reaction of a soluble acid source and an alkali monohydrogencarbonate or carbonate source. The reaction of these two general compounds produces carbon dioxide gas upon contact with water. As will be readily evident to those skilled in the art, such water-activated materials should be kept in a generally anhydrous state and with little or no absorbed moisture or in a stable hydrated form. The soluble acid sources may generally include food acids, acid and hydrite antacids such as, for example: citric, tartaric, amalic, fumeric, adipic, hydrochloric, and succinics. Carbonate sources include dry solid carbonate and bicarbonate salt such as, preferably, sodium bicarbonate, sodium carbonate, potassium bicarbonate and potassium carbonate, magnesium carbonate and the like.

The inventive particles 10 may be delivered to an intended site by various methods. For example, the inventive particles 10 may be delivered directly to the intended physiological site, or the particles may be placed in a dissolvable or erodable polymeric carrier (e.g., hard or soft gelatin capsules, starch capsules, or cellulosic capsules) and the polymeric carrier delivered to the intended site.

The polymeric carrier or capsule may be constructed in a manner and/or of a material that encourages the release of the inventive particles 10 in a specific region of the target system or environment. By way of example, the membrane thickness may be varied to expedite or retard dissolution. By way of further example, the capsules may be prepared using materials that are soluble in fluids having a pH of 3 to 5 so as to dissolve in the stomach, or with materials that are soluble in fluids having a pH of 4 to 6 so as to dissolve in the small intestine. Materials that are soluble in fluids having a pH of from 7 to 8, on the other hand, could be employed so that the capsules dissolve in the large intestine.

Once within the target system or environment, fluids having dissolved or suspended carbohydrates, lipids, metals, salts and/or toxins are drawn toward and into core 12 where they undergo a chemical reaction, are complexed, broken down, absorbed and/or adsorbed. The fluid-filled particles 10 containing the targeted materials are then removed from the site. It is noted that the inventive particles 10 enter a body and are removed or eliminated therefrom in substantially the same form.

Although the present invention has been shown and described with respect to detailed embodiments thereof, it will be understood by those skilled in the art that various changes in form and detail thereof may be made without departing from the spirit and scope of the claimed invention. For example, the inventive particles 10 or their polymeric carrier or capsule may be coated to ease swallowing, to mask taste and/or to render the particles 10 or capsule more acid resistant.

Having thus described the invention, what is claimed is:

1. Non-toxic, substantially non-degradable particles for use in reducing levels of carbohydrates, lipids, metals, salts and/or toxins in biological systems, that each comprise an optionally bifurcated core that is at least partially surrounded by a membrane or membrane-like material.

2. The non-toxic, substantially non-degradable particles of claim 1, wherein the membrane or membrane-like material is prepared from a material selected from the group of non-degradable polymers, ceramics, glasses, metals and combinations thereof.

3. The non-toxic, substantially non-degradable particles of claim 1, wherein the particles are substantially spherical particles, each having a diameter of less than about 5 millimeters.

4. The non-toxic, substantially non-degradable particles of claim 1, wherein the particles are micro-sized particles, each having a diameter ranging from about 1 to about 2,000 microns.

5. The non-toxic, substantially non-degradable particles of claim 1, wherein the particles are nano-sized particles, each having a diameter ranging from about 1 to about 1,000 nanometers.

6. The non-toxic, substantially non-degradable particles of claim 1, which are compressed into tablet form.

7. The non-toxic, substantially non-degradable particles of claim 1, which are contained within a dissolvable or erodible polymeric carrier.

8. The non-toxic, substantially non-degradable particles of claim 1, which each comprise:
(a) a solid or semi-solid core comprising materials capable of reacting or complexing with, decomposing, absorbing and/or adsorbing dissolved or suspended carbohydrates, lipids, metals, salts and/or toxins; that is at least partially surrounded by
(b) an optionally expandable membrane or membrane-like material.

9. The non-toxic, substantially non-degradable particles of claim 1, which each comprise:
(a) a hollow core; that is at least partially formed by
(b) an optionally collapsible and/or expandable membrane or membrane-like material.

10. The non-toxic, substantially non-degradable particles of claim 1, which each comprise a bifurcated core having a first portion and a second portion, wherein the bifurcated core is at least partially surrounded or formed by the membrane or membrane-like material, and wherein the first portion of the bifurcated core contains buoyancy-enhancing or effervescent agents.

11. The non-toxic, substantially non-degradable particles of claim 8, wherein the solid or semi-solid core is prepared from a material selected from the group of active materials, adsorbent materials, absorbent materials, and combinations thereof.

12. The non-toxic, substantially non-degradable particles of claim 11, wherein the solid or semi-solid core material(s) is used in combination with one or more sequestering agents.

13. The non-toxic, substantially non-degradable particles of claim 8, wherein the solid or semi-solid core is completely surrounded by the membrane or membrane-like material.

14. The non-toxic, substantially non-degradable particles of claim 8, wherein the membrane or membrane-like material is provided with at least one aperture or hole that communicates directly with the solid or semi-solid core.

15. The non-toxic, substantially non-degradable particles of claim 14, wherein the at least one aperture or hole is provided with an erodable stopper or plug that serves to prevent or retard capillary movement of fluid into or out of the solid or semi-solid core.

16. The non-toxic, substantially non-degradable particles of claim 14, wherein the at least one aperture or hole contains a swellable material that swells or increases in size when exposed to fluids found in a target environment, thereby containing or sequestering acquired fluids within the solid or semi-solid core.

17. The non-toxic, substantially non-degradable particles of claim 9, wherein the hollow core is completely surrounded by the membrane or membrane-like material.

18. The non-toxic, substantially non-degradable particles of claim 9, wherein the membrane or membrane-like material is provided with at least one aperture or hole that communicates directly with the hollow core.

19. The non-toxic, substantially non-degradable particles of claim 18, wherein the at least one aperture or hole is provided with an erodable stopper or plug that serves to prevent or retard capillary movement of fluid into or out of the hollow core.

20. The non-toxic, substantially non-degradable particles of claim 18, wherein the at least one aperture or hole contains a swellable material that swells or increases in size when exposed to fluids found in a target environment, thereby containing or sequestering acquired fluids within the hollow core.

21. The non-toxic, substantially non-degradable particles of claim 9, wherein a partial vacuum is formed within each particle.

22. A method of reducing levels of carbohydrates, lipids, metals, salts and/or toxins in biological systems, which comprises: delivering a plurality of non-toxic, substantially non-degradable particles to a biological system, wherein each particle comprises an optionally bifurcated core that is at least partially surrounded by a membrane or membrane-like material.
